(19) United States
(12) Patent Application Publication
(10) Pub. No.: US 2019/0161732 A1
(43) Pub. Date: May 30, 2019

(54) METHOD OF USING CHITOSANFOR REJUVENATING CELLS AND COMPOSITION THEREOF

(71) Applicant: National Taiwan University, Taipei City (TW)

(72) Inventors: TAI-HORNG YOUNG, Taipei City (TW); CHING-WEN TSAI, Taipei City (TW); JYH-HORNG WANG, Taipei City (TW)

(21) Appl. No.: 15/925,732

(22) Filed: Mar. 19, 2018

(30) Foreign Application Priority Data
Nov. 27, 2017 (TW) 106141236

(51) Int. Cl. C12N 5/077; C12N 5/071

(52) U.S. Cl. CPC C12N 5/0656; C12N 2501/90; C12N 5/0629

(57) ABSTRACT

The present application provides a method for rejuvenating cells, characterized in that providing a cell to culture in a composition for a suitable period of time of time, wherein the composition is obtained by adding a chitosan solution to a cultural environment. The present application further provides a composition for rejuvenating cells, comprising a chitosan solution in a cultural environment.

### CROSS-REFERENCE TO RELATED APPLICATIONS

This application claims the priority of Republic of China Patent Application No. 106141236 filed on Nov. 27, 2017, in the State Intellectual Property Office of the R.O.C., the disclosure of which is incorporated herein by reference.

### BACKGROUND OF THE INVENTION

### Field of the Invention

The present application relates to a method and a composition for rejuvenating cells, and particularly relates to a method for rejuvenating aged cells using a composition containing chitosan.

### Descriptions of the Related Art

Senescence (senility), also known as aging, usually refers to the functional decline, the decrease of body stability, structural center component degradation, and the irreversible phenomenon of the tendency to death with age under normal circumstances after the biologicals developing to maturity. Aging and death are the basic phenomena of life. The aging process takes place in different levels of biology such as overall level, population level, individual level, cell level, molecular level, etc. Life must be constantly renewed, races have to constantly multiply. This process is carried out in the contradiction between life and death. At least from a cellular level, death is inevitable.

Aging is a universal law in the biological world. Cells, as the basic unit of biological organisms, also continue regenerating, aging and death. Aging is a process, and the length of the process is the life-span of the cell which varies with the type of tissue and be affected by environmental conditions too. There is maximum division number of somatic cells in higher animals, the cell dies once the division reaches the number. The maximum number of the cell division varies from animal to animal, and human cell is 50 to 60 times. In general, the maximum division number of cells is proportional to the average life-span of animals Series of changes would occur when cell senescence, such as the decrease of water content, accumulation of senile pigment-lipofuscin, the decrease of enzyme activity, slow down of metabolism rate, and so on. There are many theories about cell senescence, including genetic factor theory, cell damage theory, biological macromolecule senescence theory, etc., but all of them cannot solve the problem satisfactorily.

p53 and pRB proteins are two key tumor suppressor factors and play a decisive role in senescence induction. The two general aging signal pathways refer to the pRB signal pathway and the p53 signal pathway.

The expressions of aging pathways of human cells are two parallel pathways, which will make it difficult for cells to bypass the aging process and thereby inhibit oncogenesis. However, this concept has been challenged by some experiments in recent years, such as human lung fibroblasts can avoid the aging process by inactivating p53 or pRB through somatic homologous recombination technology.

As for the role of p53 and pRB proteins, there is now a more accepted theory that the p53/p21 pathway refers to the aging process caused by telomere dysfunction and DNA damage; the p16/pRB pathway refers to the aging process caused by the expression of oncogenes, chromatin breaks, a variety of coercion, and so on. But now the molecular markers of these two pathways have not been found, and the importance of the two pathways in cells varies from species to species, from tissue to tissue. Therefore, the understanding of the aging pathway is in the process of deepening.

Since cell therapy is a new therapeutic trend, cell transplantation after culture and proliferation from the extracted autologous cells can provide abundant growth factors and ideal physical support for the treatment of affected parts. The elderly or damaged aging cells, however, are prone to poor therapeutic effects due to the inadequate cell proliferation or the low migration ability.

At present, most of the methods to prevent aging are to place aged cells in a younger environment or to treat them with a low oxygen concentration. In the clinical application of medicine, ACR (Autologous Cell Rejuvenation) of PRR (Platelet Rich Plasma) technology is used to obtain the high concentration of autologous growth factors, to stimulate tissue repair, to effectively delay aging, and to further achieve the effect of rejuvenation. Academically, however, placing aged cells in a young environment may have rejection because the young serum and ECM come from allogeneic sources. In clinical medicine, the opportunities for actual clinical application would be lower if cells were treated with the low oxygen concentration, because while human beings in the environment of oxygen concentration less than 18% would lead to the inadequate intake of oxygen, an abnormally low level of oxygen partial pressure in the blood, and hemoglobin in the unsaturated state. Cells of all parts of tissues would change and show the corresponding symptoms of hypoxia due to the lack of oxygen. Although the rejuvenation techniques in current clinical is to use autologous plasma which is not rejective and is rich of growth factors, the growth factors are unstable and may also trigger tumor formation due to the high concentration of growth factors.

In addition, Republic of China Patent No. I550088 discloses a method for rejuvenating aged cells, comprising following steps: (a) dissolving a biodegradable polymer material into a solution; (b) producing a carrier from the solution; and (c) culturing the aged cells on the carrier for 1-14 days to rejuvenate the aged cells; wherein biocompatible polymer material can be polyvinyl alcohol, poly(hydroxyethyl methacrylate), chitosan, methyl cellulose, agar, hyaluronic acid or combination thereof. The carrier was prepared by adding the solution of the biocompatible macromolecular material to a cell culture plate and drying at 60° C. for 24 hours, then neutralizing with sodium hydroxide solution to about pH 7; subsequently, cleaning the cell culture plate with Milli-Q water and exposing to UV light overnight. The method is to make the aged cells into a substantially spherical shape by using special materials so that the substantially spherical ball center to the ball edge results in varying degrees of hypoxia to render the aged cells to have the effect of rejuvenation. However, the preparation process is complicated because the carrier needs to be prepared from the biocompatible macromolecule material in advance in the process and then it needs to be carried out steps of alkali neutralization; moreover, the concentration of the biocompatible macromolecule material cannot be adjusted easily. Therefore, there is still a need for a simple, convenient and easy-to-use method for rejuvenating aged cells.

### SUMMARY OF THE INVENTION

In view of the problems existing in the prior art, the present application provides a method and a composition for delaying cell aging, characterized in that providing a cell to culture in a composition for a suitable period of time, wherein the composition is obtained by adding a chitosan solution to a cultural environment.

In an embodiment, the chitosan solution further comprises an acetic acid solution, a phosphate buffer, water or a mixture thereof. Preferably, the concentration of the acetic acid solution is 0.05 wt % to 75 wt %. Preferably, the chitosan concentration of the composition is 10 μg/ml to 200 μg/ml.

In an embodiment, the cell is selected from the group consisting of fibroblast, adipose-derived stem cell, mesenchymal stem cell, cruciate ligament cell, synoviocyte, and keratinocyte.

In an embodiment, the pH value of the composition is 6 to 8. Preferably, the pH value of the composition is 6 to 7.

In an embodiment, the suitable period of time is more than 1 day, preferably 1-7 days.

The present application provides a method and a composition for rejuvenating cells, which doesn't need to prepare chitosan to a carrier in advance and eliminates the carrier preparation processes and steps of alkali neutralization after the carrier prepared. The present application reaches the effect of rejuvenating cells by directly adding the chitosan solution into the medium so that the chitosan concentration can be freely adjusted to achieve the desired effect, solving the problems of the prior art and having significant beneficial effects.

The present application can be applied to various technical fields of cell culture in particular to the cell transplantation after culture and proliferation from the extracted autologous cells in cell therapy. Even the elderly or damaged aging cells can provide abundant growth factors and ideal physical support for the treatment of affected parts after rejuvenating the cells through the method and the composition of the present application.

The details of the invention are set forth in the following description, which is to be regarded as illustrative methods and materials only, and not restrictive. Other similar or equivalent methods and materials described herein to practice or test the present invention should be regarded as the scope of the resent application. In the specification and the appended claims, the singular form includes the plural as well unless the context clearly indicates otherwise. Unless otherwise defined, all technical and scientific terms used herein have the same meaning as generally understood as one having ordinary skill in the art of the present invention.

### BRIEF DESCRIPTION OF THE DRAWINGS

The above and other aspects, features and other advantages of the present invention will be more clearly understood from the following detailed description taken in conjunction with the accompanying drawings, in which:

FIG. 1A˜FIG. 1D shows a staining result of the SA-β-gal assay of human foreskin fibroblasts, which the cells were cultured on TCPS in the compositions containing different chitosan concentrations. FIG. 1A The medium without the addition of chitosan solution. FIG. 1B The composition containing 20 μg/ml chitosan. FIG. 1C The composition containing 60 μg/ml chitosan. FIG. 1D The composition containing 100 μg/ml chitosan.

FIG. 2 shows a semi-quantitative result of the SA-beta-gal assay of human foreskin fibroblasts, which the cells were cultured on TCPS in the compositions containing different chitosan concentrations.

FIG. 3 shows a BrdU assay result of human foreskin fibroblasts, which the cells were cultured on TCPS in the compositions containing different concentrations of chitosan.

FIG. 4A to FIG. 4D shows a staining result of the SA-β-gal assay of human foreskin fibroblasts, which the cells were cultured on PVA and pHEMA in the composition containing 100 μg/ml chitosan. FIG. 4A The cells were cultured on PVA without the addition of the chitosan solution in medium. FIG. 4B The cells were cultured on PVA in the composition containing 100 μg/ml chitosan. FIG. 4C The cells were culture on pHEMA without the addition of chitosan solution in the medium. FIG. 4D The cells were cultured on pHEMA in the composition containing 100 μg/ml chitosan.

FIG. 5 shows a semi-quantitative result of the SA-beta-gal assay of human foreskin fibroblasts, which the cells were cultured on PVA and pHEMA in the composition containing 100 μg/ml chitosan.

FIG. 6 shows a BrdU assay result of human foreskin fibroblasts, which the cells were cultured on PVA and pHEMA in the composition containing 100 μg/ml chitosan.

FIG. 7 shows a result of TGF-β expression of human foreskin fibroblasts using the western blot assay, which the cells were cultured on TCPS, PVA, and pHEMA in the composition containing 100 μg/ml chitosan.

FIG. 8 shows a semi-quantitative result of TGF-β expression of human foreskin fibroblasts using the western blot assay, which the cells were cultured on TCPS, PVA, and pHEMA in the composition containing 100 μg/ml chitosan.

FIG. 9A˜FIG. 9C is shows a staining result of the SA-β-gal assay of human foreskin fibroblasts, which the cells were cultured on TCPS in the medium containing 100 μg/ml PVA or in the medium containing 100 μg/ml pHEMA. FIG. 9A The medium without the addition of PVA or pHEMA solution. FIG. 9B The medium with the addition of PVA solution. FIG. 9C The medium with the addition of pHEMA

### SOLUTION

FIG. 10 shows a semi-quantitative result of the SA-beta-gal assay of human foreskin fibroblasts, which the cells were cultured on TCPS in the medium containing 100 μg/ml PVA or in the medium containing 100 μg/ml pHEMA.

FIG. 11 shows a BrdU assay result of human foreskin fibroblasts, which the cells were cultured on TCPS in culture in the medium containing 100 μg/ml PVA or in the medium containing 100 jug/ml pHEMA.

FIG. 12A˜FIG. 12D shows a staining result of the SA-β-gal assay of human foreskin fibroblasts, which the cells were cultured in the compositions of the present application at different pH values. FIG. 12A The pH 7.7 medium without the addition of chitosan solution. FIG. 12B The pH 7.7 composition of the present application. FIG. 12C The pH 6.9 medium without the addition of chitosan solution. FIG. 12D The pH 6.9 composition of the present application.

FIG. 13 is a semi-quantitative result of the SA-beta-gal assay of human foreskin fibroblasts, which the cells were cultured in the compositions of the present application at different pH values.

FIG. 14 shows a BrdU assay results of human foreskin fibroblasts, which the cells were cultured in the compositions of the present application at different pH values.

FIG. 15A˜FIG. 15F shows a staining result of the SA-beta-gal assay of human foreskin keratinocytes, which the cells were cultured on TCPS in the compositions containing different chitosan concentrations. FIG. 15A The medium with addition of the chitosan solution. FIG. 15B The composition containing 20 μg/ml chitosan. FIG. 15C The composition containing 40 μg/ml chitosan. FIG. 15D The composition containing 60 μg/ml chitosan. FIG. 15E The composition containing 80 μg/ml chitosan. FIG. 15F The composition containing 100 μg/ml chitosan.

FIG. 16 shows a result of RB, p53, and p21 expression of human foreskin fibroblasts using western blot assay, which the cells were cultured on TCPS in the compositions containing different chitosan concentrations.

### DETAILED DESCRIPTION OF THE PREFERRED EMBODIMENT

Reference will be made in detail description to the exemplary embodiments and drawings for being more readily understood to the advantages and features of the present invention, as well as the methods of attaining them. However, the present invention may be carried out in many different forms and should not be construed as limited to the embodiments set forth herein. Conversely, these embodiments are provided to render the present disclosure to be conveyed the scope of the present invention more thoroughly, completely, and fully to one having ordinary skill in the art of the present invention. Moreover, the present invention would be defined only by the appended claims. As used herein, the term “and/or” includes any and all combinations of one or more of the associated listed components.

Unless otherwise defined, all terms (including technical and scientific terms) used herein have the same meaning as generally understood by one having ordinary skill in the art of the present invention. It will be more understandable that, for example, the terms defined in commonly used dictionaries should be understood to have meanings consistent with the contents of the relevant fields, and would not be interpreted overly idealized or overly formal unless clearly defined herein. As described in the present specification, a range of values is used as a shorthand to describe each and every numerical value in the range, and any number within that range may be chosen as the end-value of that range.

The present application provides a method and a composition for rejuvenating cells, characterized in that providing a cell to culture in a composition for a suitable period of time, wherein the composition is obtained by adding a chitosan solution to a cultural environment.

In an embodiment, the chitosan solution further comprises an acetic acid solution, a phosphate buffer, water or a mixture thereof. Preferably, the concentration of the acetic acid solution is 0.05 wt % to 75 wt %. Preferably, the chitosan concentration of the composition is 10 μg/ml to 200 μg/ml.

In an embodiment, the cell are selected from the group consisting of fibroblast, adipose-derived stem cell, mesenchymal stem cells, cruciate ligament cell, synoviocyte, and keratinocyte.

In an embodiment, the pH value of the composition is 6 to 8. Preferably, the pH value of the composition is 6 to 7.

In an embodiment, the suitable period of time for the cell cultured in the composition is more than 1 day, preferably 1-7 days.

After culturing the cell using the method and the composition of the present application, whether the cell rejuvenated is determined through SA-β-gal assay, BrdU assay and western blot assay (detecting TGF-β, RB, p53, and p21). However, it should be noted that the determining methods and the detected types of proteins utilized in the present invention for determining whether cells rejuvenated are merely illustrative, not restrictive. Many of the currently well-developed test methods or related protein types for determining rejuvenation can be used in the present application.

As used herein, the term “SA-β-gal assay” refers to a test of senescence β-galactosidase staining, which is a test to stain senescent cells or tissues based on the increase of the senescence-associated β-galactosidase activity while senescence. Aging conditions of cells or tissues can be observed in ordinary optical microscope, and it can also be used in the biopsy for aging test. All the SA-β-gal assays of the examples herein were performed by the following steps. First, cells were fixed in 4% formaldehyde and incubated with X-gal in SA β-gal staining solution at pH 6 in the absence of CO₂ at 37° C. Next, the stained cells were photographed, and the percentages of cells with blue sedimentation in the cytoplasm were counted. For calculating the percentages of cells stained SA β-gal, at least 400 cells were calculated from ten randomly selected fields for each case.

As used herein, the term “BrdU assay” refers to the detection of cells in S phase with bromodeoxyuridine (BrdU), which replaces thymidine to incorporate into DNA of S-phase cells when being added to the cultured cells so as to determine the cell's proliferation ability. All the BrdU assays of the examples herein were performed by the following steps. First, cells were seeded and well attached to a culture plate of 100 μl/well before adding BrdU. Next, 0.2 μl of BrdU was added to each well, and the cells were incubated at 37° C. for 12 hours. After BrdU incorporation, the cells were immobilized using the fixing solution (Millipore) for 30 minutes to inactivate DNA and then the cells were washed for 3 times using wash buffer (Millipore). After that, 100 μl of anti-BrdU monoclonal antibody was added to each well, and the cells were incubated at room temperature for 1 hour. 100 μl of Goat anti-Mouse IgG peroxidase conjugate, which diluted in 1:2000, was then added to each well, and the cells were incubated at room temperature for 30 minutes in this step. Further, the cells were washed for 3 times using the wash buffer, and incubated at room temperature in the dark for 30 minutes after the tetramethylbenzidine (TMB) being added. After 30 minutes, results of the culture plate was read using an ELISA reader at the wavelength of 370/690 nm.

As used herein, the term “western blot assay” refers to analyze a biological detection technology of specific protein detection for obtaining information of the expression condition of a specific protein in the analyzed cells or tissues by analyzing the colored position and the color depth according to the principle, which a specific antibody can specifically bind to the antigen protein to color the sample. All the western blot assays of the examples herein were performed by the following steps. First, cells were lysed with the lysis buffer and centrifugated at 16,000 g for 5 minutes at 4° C. to collect the soluble protein. Next, the supernatant was transferred to laemmli buffer and heated at 95° C. for 5 minutes. Following that, the protein was extracted through separated by SDS-PAGE and transferred onto a PVDF membrane. After blocking, The membrane was incubated with primary antibodies, such as p53 antibody (Merck OP09; PAb1891; 1:200), p21 antibody (Cell Signaling #2946; DSC60; 1:2000), pRB antibody (Merck OP66; LM95.1; 1:100), TGF-β antibody (Cell signaling 3709; 56E4; 1:1000), and GAPDH antibody (Abcam ab22555; polyclone; 1:10000), at 25° C. for 2 hours and then was placed at 4° C. overnight. Afterwards, the PVDF membrane was incubated with the peroxidase-labeled secondary antibody at 25° C. for 2 hours then detected by the chemiluminescence detection system. All the western blot assays were repeated at least 3 times.

As used herein, the term “cultural environment” refers to the environment that provides the required components for the growth while cells culture, such as amino acids, glutamine, vitamins, glucose, galactose, sodium bicarbonate, salts, HEPES, phosphate buffer, serum, fetal bovine serum, bovine pituitary extract (BPE), antibiotics, and so on. Conventional or commercially available culture broths or medium of cell/tissue can be used as the cell cultural environment such as, but not limited to, medium KSFM, DMEM, RPMI 1640, MEM, F12, McCoy's, IMDM, M-199, MCDB 131, L-15, Ham's F-10, Ham's F-12, E medium, Opti-MEM, and so on.

As used herein, the term “rejuvenating” refers to delay cell aging and enhance cell proliferation as well as enhance cellular functionality to restore cells to a young and healthy state.

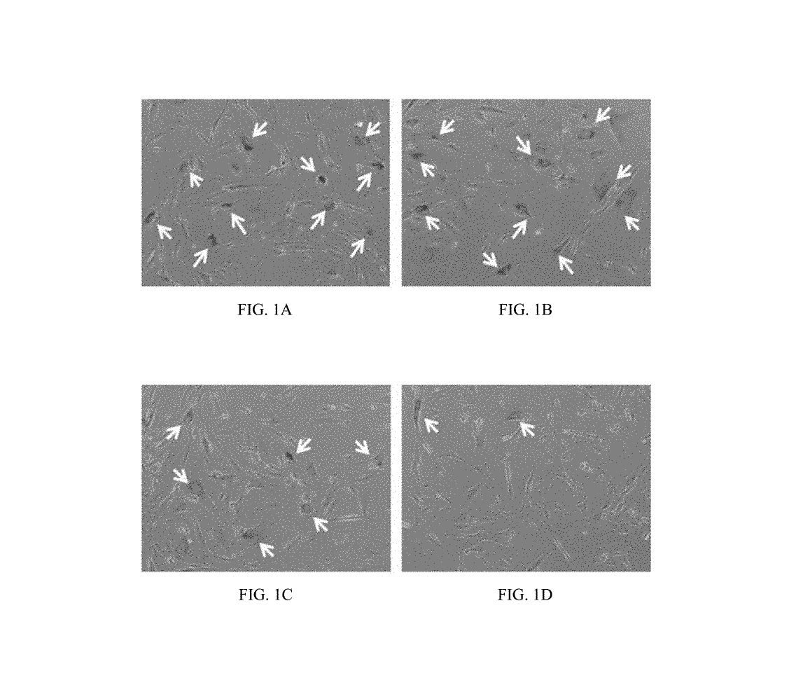

The following examples will illustrate the experiment procedure and results of the method and the composition for rejuvenating cells provided by the present invention. The test results are shown in FIG. 1 to FIG. 16; however, the test results of the examples are merely illustrative and are not intended to limit the scope of the present invention.

### Example 1

The cells referred to in the following examples are all fibroblasts unless otherwise specified. However, the usage of the fibroblast are only illustrative but not restrictive, others such as adipose-derived stem cell, mesenchymal stem cell, cruciate ligament cell, synovial cell, keratinocyte, or a combination thereof can be used in the present invention. The fibroblasts used in the examples of the present application were obtained through isolating the human foreskin fibroblasts from specimens of donors (patients who needed to undergo circumcision).The ages of donors were about fifteen to thirty, and five strains were used in the examples of the present application. The study protocol was approved by the Institutional Review Board of the National Taiwan University Hospital. The protocols of the cells obtaining and culturing are described below. The specimens were placed in Dulbecco's Modified Eagle's Medium (DMEM), and were separated the epidermis from the dermis using 1 mg/ml of dispase. Next, the dermis was cut into squares 2-3 mm in sides and was placed on the medium of 100 mm. The dermis was incubated in DMEM with 10% fetal bovine serum (FBS; GIBCO) and 1% penicillin/streptomycin at 37° C. with 5% CO₂ until the fibroblasts had out-migrated from the dermis. The fibroblasts are then subcultured in the medium.

### Example 2

The human foreskin fibroblasts obtained in Example 1 were seeded on a 6-well TCPS with a cell density of about 5×10³/cm², and then the chitosan solution was added to the medium (DMEM+10% FBS) and mixed to obtain the composition of the present application, while the chitosan concentration of the composition was 20 μg/ml, 60 μg/ml, and 100 μg/ml, respectively. The SA-β-gal assay and the BrdU assay were carried out after the cells were cultured at 37° C. with 5% CO₂ for 3 days. (Stock solution is 3% chitosan solution, which means 30 mg/ml=30 μg/μl. Taking 100 μg/ml group as an example, 2 ml needs 200 μg of chitosan molecule, so it will take 6.67 μl of 3% chitosan solution to add to the medium of 2 ml.)

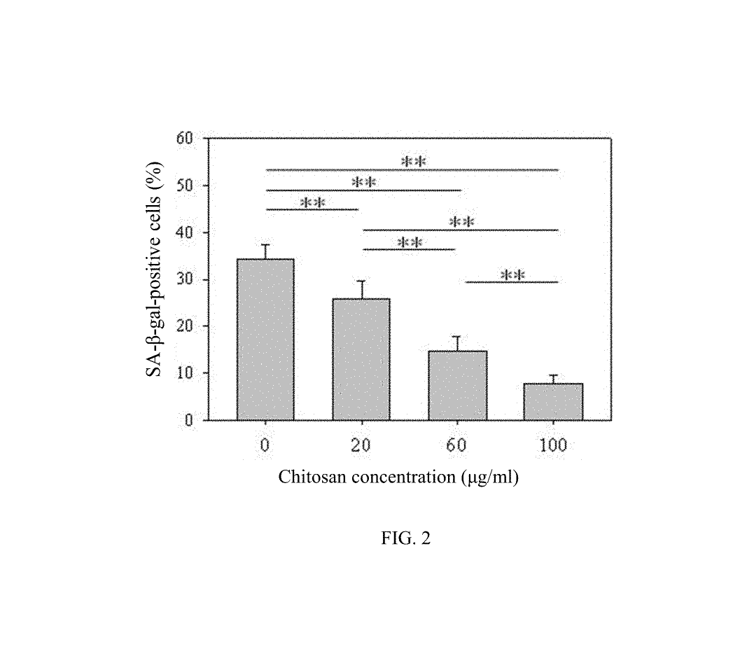

Please refer to FIG. 1A to FIG. 1D. FIG. 1A to FIG. 1D shows the staining result of the SA-β-gal assay of the cells cultured in different chitosan concentrations of the composition. As can be seen in FIG. 1A to FIG. 1D that the blue stained in the cells are SA-β-gal (aged cells would be stained), and most of the cells in the medium without chitosan solution (FIG. 1A) are stained blue. Whereas for the cells cultured in the medium containing 20 μg/ml (FIG. 1B), 60 μg/ml (FIG. 1C), and 100 μg/ml (FIG. 1D) chitosan, the number of the SA-β-gal-positive cells decrease significantly with the increase of the chitosan concentration of the composition. In addition, as can be easily seen in the semi-quantitative results of the SA-β-gal assay of FIG. 2 that the proportion of the SA-β-gal-positive cells significantly decreases with the increase of the chitosan concentration of the composition (p<0.01).

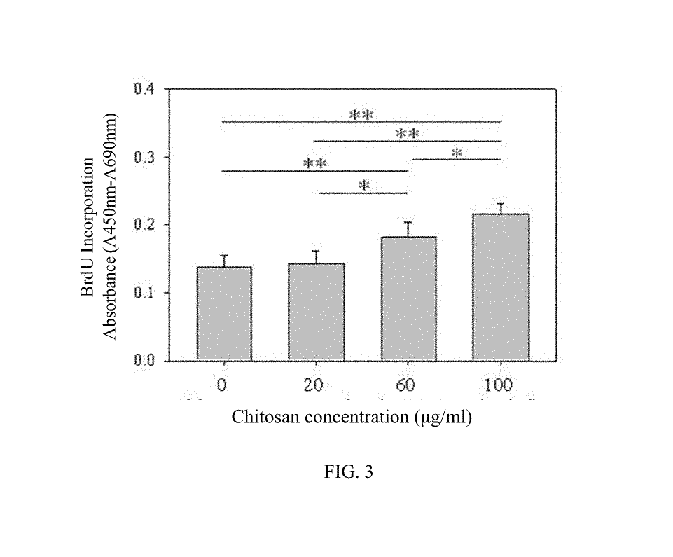

Please refer to FIG. 3. FIG. 3 shows the BrdU assay result of the cells cultured in different chitosan concentrations of the compositions. As can be seen in FIG. 3 that the higher the chitosan concentration of composition, the higher BrdU absorbance of the cells is detected (p<0.01 and p<0.05).

This example found that the cells cultured by the method and the composition of the present application having the result of a significant decrease of stained cells in the SA-β-gal assay compared with the cells cultured without the addition of chitosan solution, which shows the senescent or aged cells are rejuvenated. Moreover, the cells cultured by the method and the composition of the present application having a higher BrdU absorbance as well in the BrdU assay, indicating the cells having a significant increase of proliferation rate of cell population compared with the cells cultured without the addition of chitosan solution. Besides, the effect of rejuvenating cell improves with the increase of the concentration of the added chitosan solution.

### Example 3

The human foreskin fibroblasts obtained in Example 1 were seeded on a 6-well TCPS coated with PVA and pHEMA at a cell density of about 5×10³/cm². The method of coating PVA and pHEMA onto TCPS followed the preparation method disclosed in the Republic of China Patent No. I550088, which adding PVA and pHEMA solution to cell culture plates and drying at 60° C. for 24 hours, then neutralizing with sodium hydroxide solution to about pH 7; subsequently, cleaning the cell culture plate with Milli-Q water and exposing to UV light overnight. After that, the chitosan solution was added to the medium (DMEM+10% FBS) and mixed to obtain the composition of the present application, while the chitosan concentration of the composition was 100 μg/ml. The SA-β-gal assay and the BrdU assay were carried out after the cells were cultured at 37° C. with 5% CO₂ for 3 days as well.

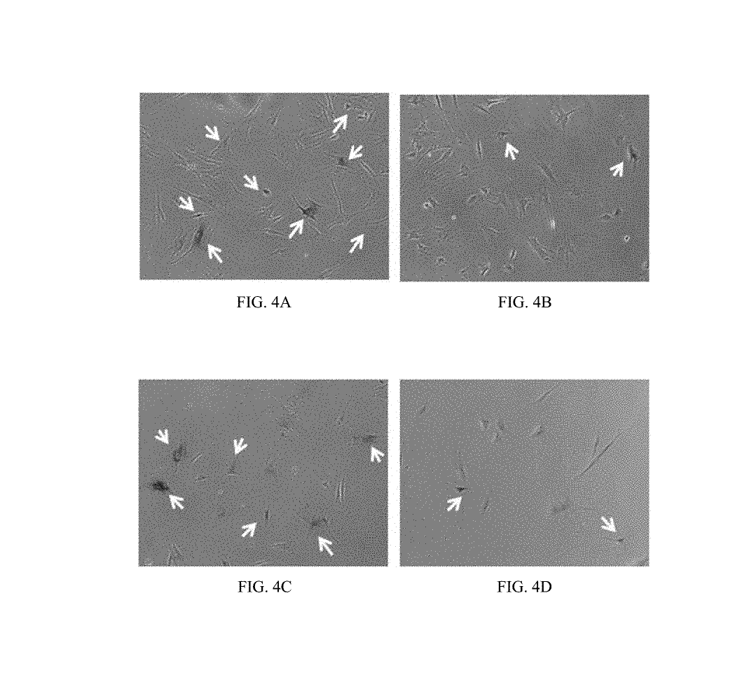

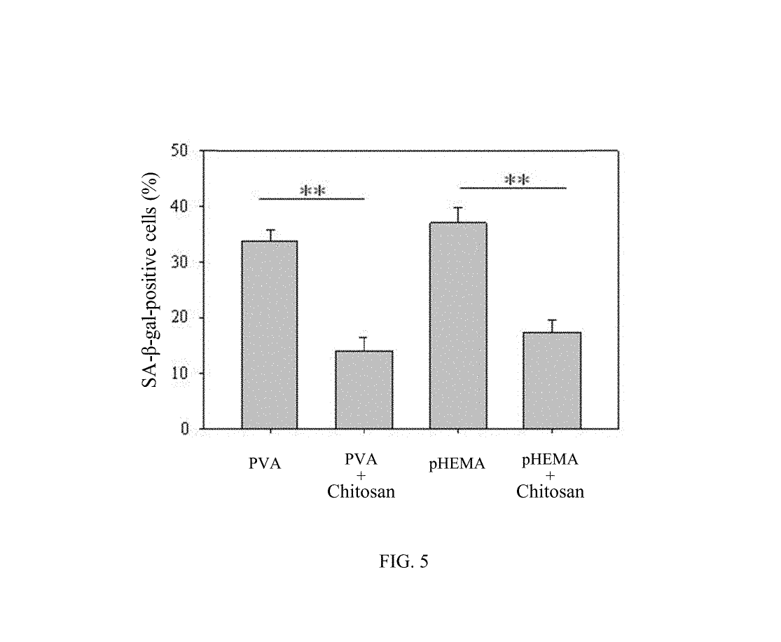

Please refer to FIG. 4A to FIG. 4D. FIG. 4A to FIG. 4D shows the staining result of the SA-β-gal assay of human foreskin fibroblasts, which the cells were cultured on PVA and pHEMA in the composition containing 100 μg/ml chitosan. As can be seen clearly in FIG. 4A to FIG. 4D, most of the cells cultured on PVA in the medium without the addition of chitosan solution (FIG. 4A) are stained blue, whereas the number of the SA-β-gal-positive cells in the medium with the addition of chitosan solution (FIG. 4B) decrease significantly. For cultured on pHEMA, similarly, the number of the SA-β-gal-positive cells of the cells in the medium with the addition of chitosan solution (FIG. 4D) decrease significantly compared with those of the cells without the addition of chitosan solution (FIG. 4C). In addition, as can be easily seen in the semi-quantitative results of the SA-β-gal assay of FIG. 5 that the proportion of the SA-β-gal-positive cells of the cells cultured on TCPS coated with whichever PVA or pHEMA significantly decreases compared with those of the cells without the addition of chitosan solution (p<0.01).

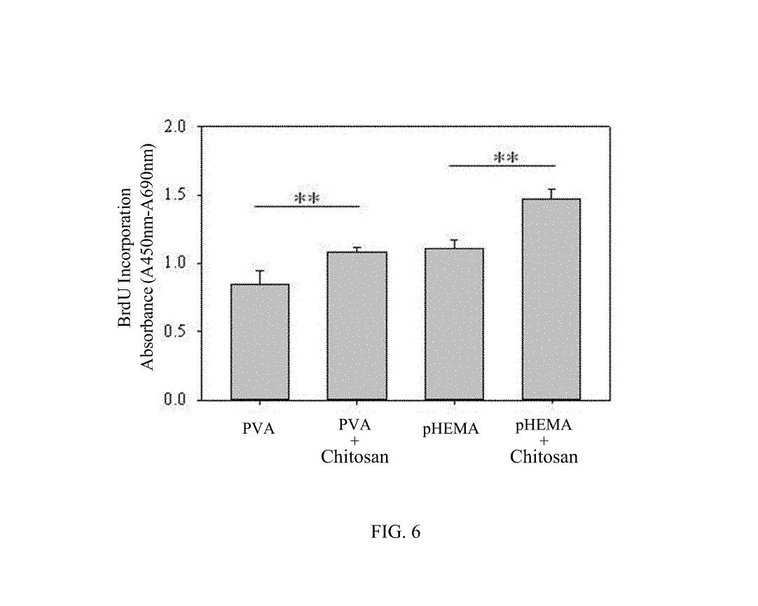

Please refer to FIG. 6. FIG. 6 shows the BrdU assay result of human foreskin fibroblasts, which the cells were cultured on PVA and pHEMA in the composition containing 100 μg/ml chitosan. As can be seen in FIG. 6, the BrdU absorbance of the cells cultured on TCPS coated with whichever PVA or pHEMA in the medium with the addition of chitosan solution increases significantly compared with those of the cells without the addition of chitosan solution.

In addition, the cells cultured on TCPS, PVA and pHEMA in the composition containing 100 μg/ml chitosan were determined for the expression of TGF-β protein using the western blot assay.

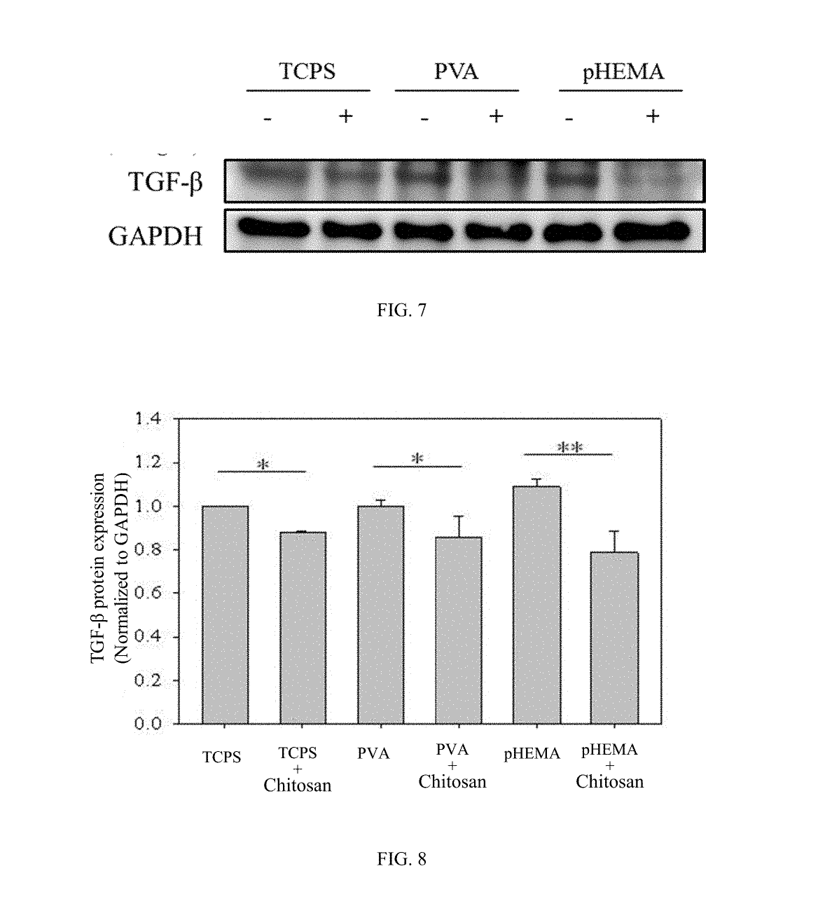

Please refer to FIG. 7, “−” indicates the control group which the medium without the addition of chitosan solution, and “+” indicates the group that the medium with the addition of the chitosan solution to make the composition containing 100 μg/ml chitosan. As can be seen in FIG. 7, the TGF-β expression of the cells cultured on whichever TCPS, PVA or pHEMA in the medium with the addition of chitosan solution is lower than the control group. Further, as can be seen in the semi-quantitative results of FIG. 8 that there is a significant decrease of the TGF-β expression in the cells of medium with the addition of chitosan solution comparing with the control group (p<0.01 and p<0.05).

This example found that the method and the composition of the present application can be applied to different cell culture carriers or substrates and achieve the same effect of rejuvenating cells. Disclosed in the example, besides, the effects of the method and the composition of the present application are significantly better than the method described in the Republic of China Patent No. I550088 by comparing the experiments in this example. Even though I550088 discloses to culture cells on the carrier coated with PVA and pHEMA can rejuvenate aged cells, the addition of chitosan solution in the medium has more pronounced and superior effect of rejuvenating aged cells.

### Example 4

The human foreskin fibroblasts were cultured on TCPS then the PVA solution and the pHEMA solution were respectively added into the medium (DMEM+10% FBS), and mixed to form the medium containing 100 μg/ml PVA and the medium containing 100 μg/ml pHEMA. The SA-β-gal assay and the BrdU assay were carried out after the cells were cultured at 37° C. with 5% CO₂ for 3 days.

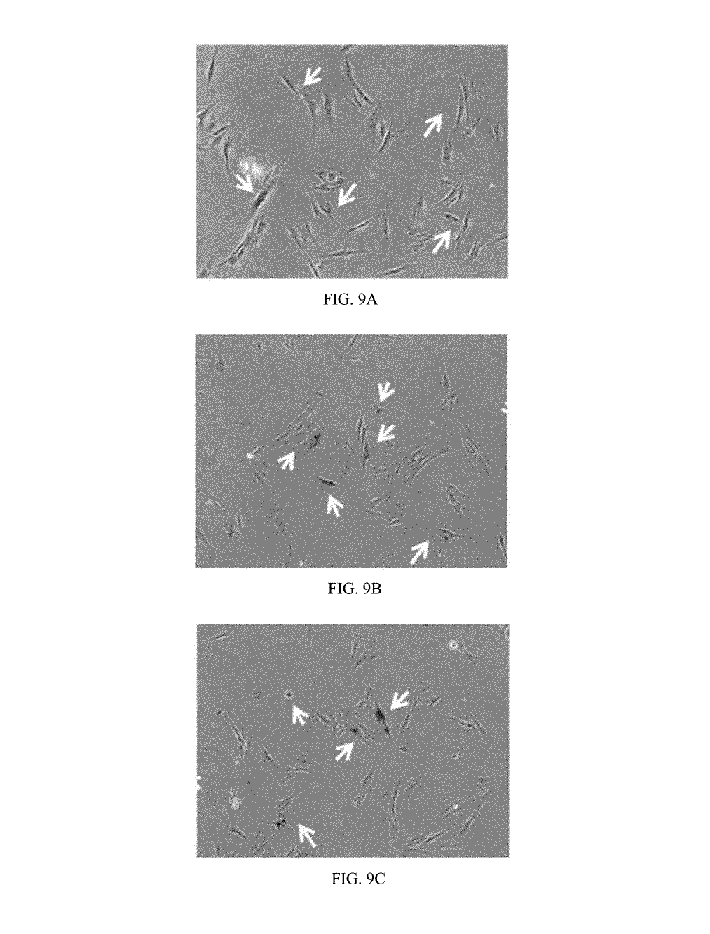

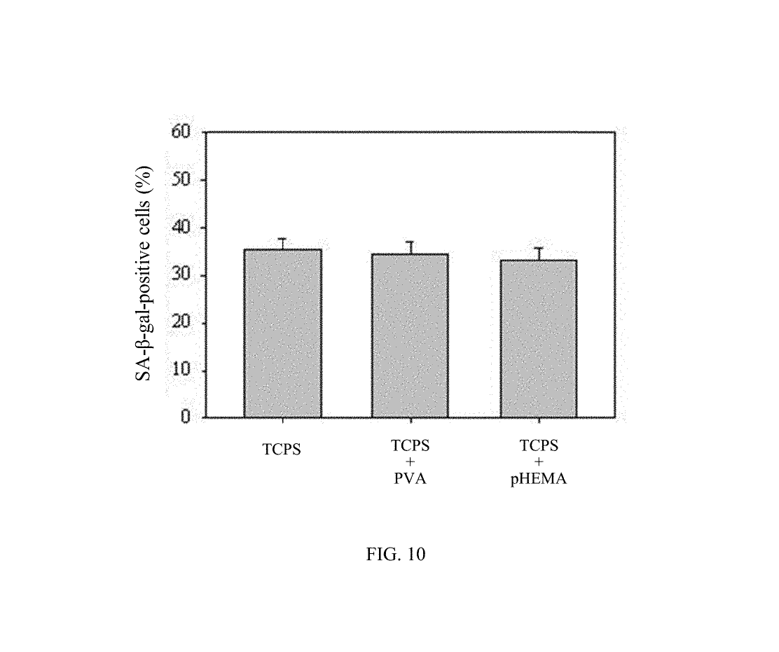

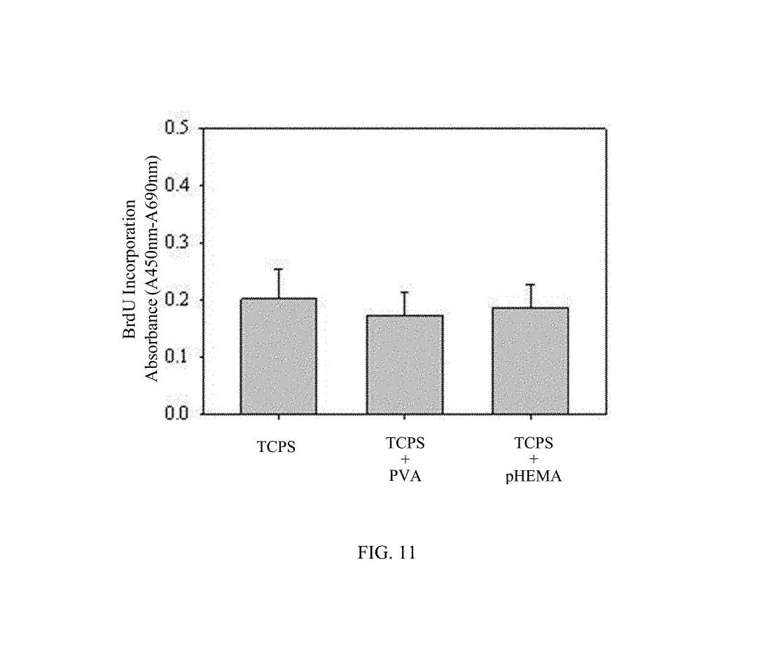

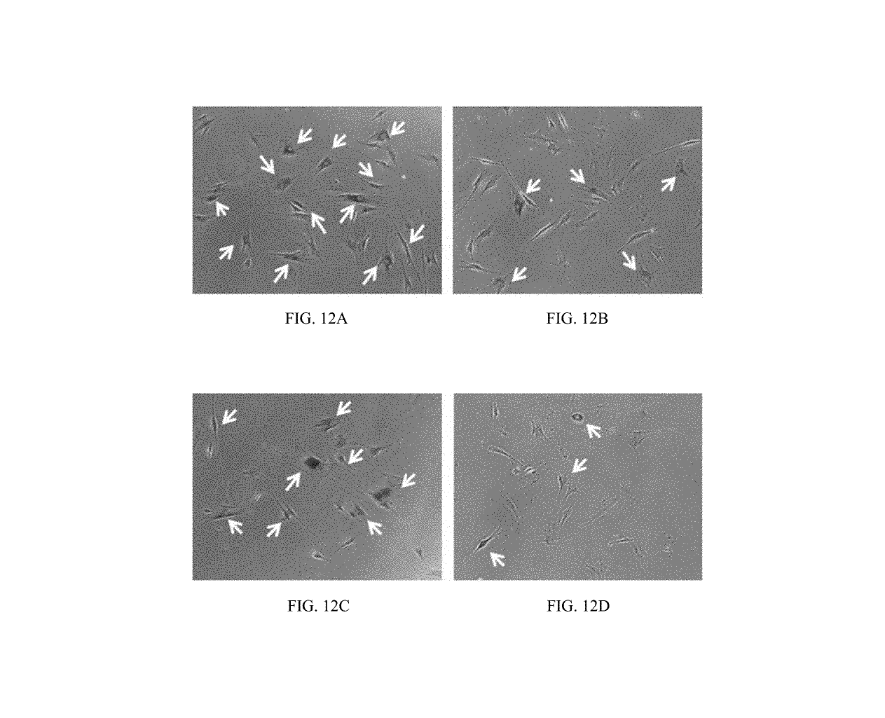

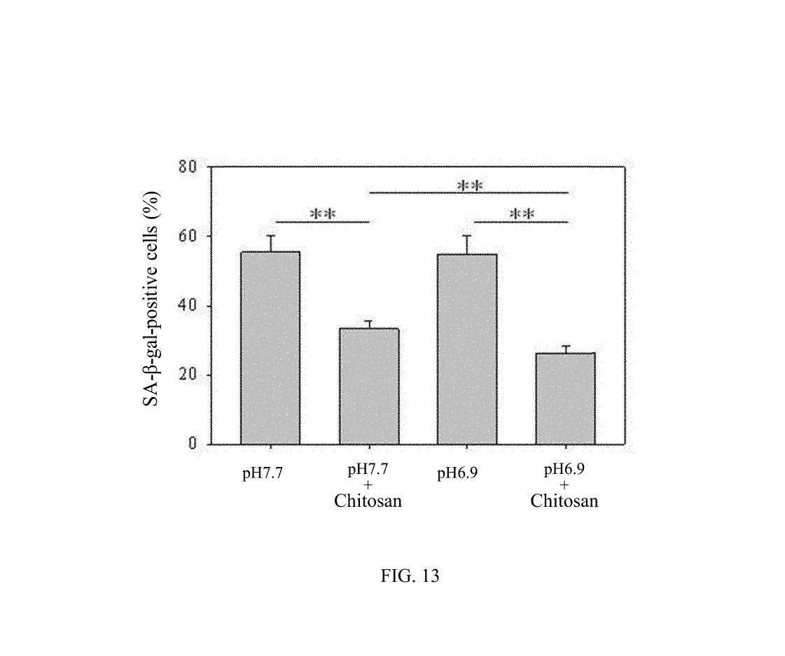

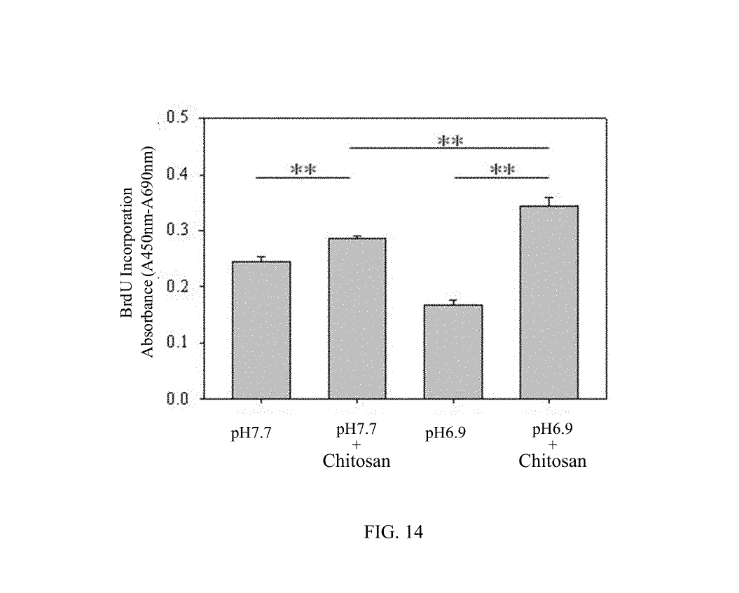

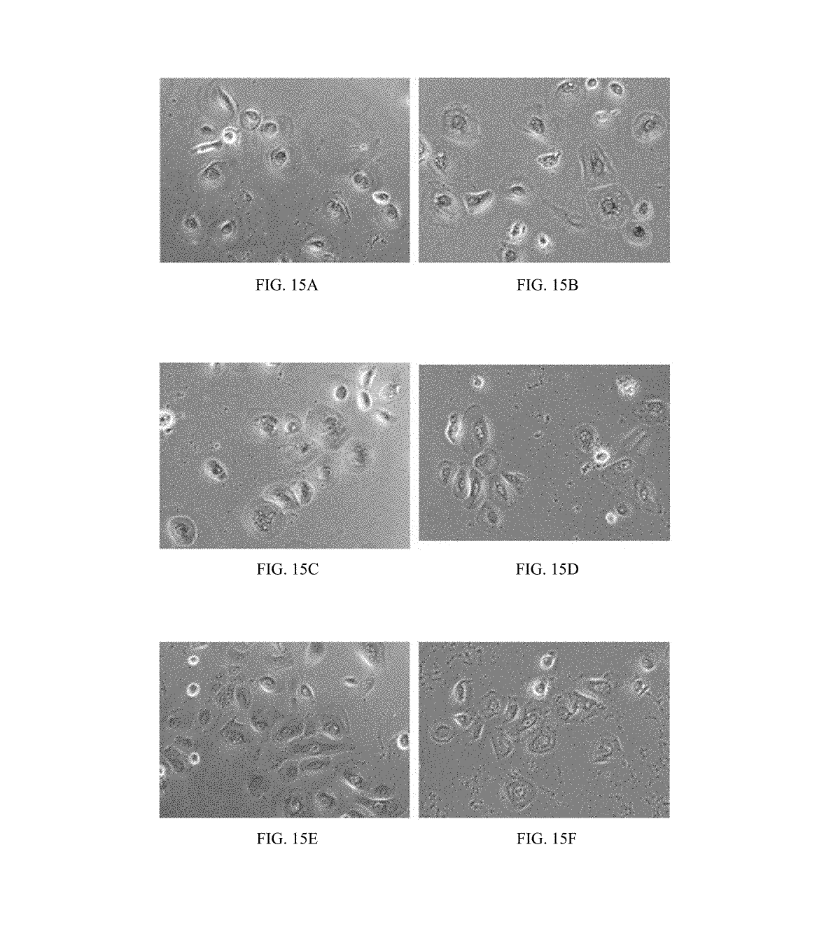

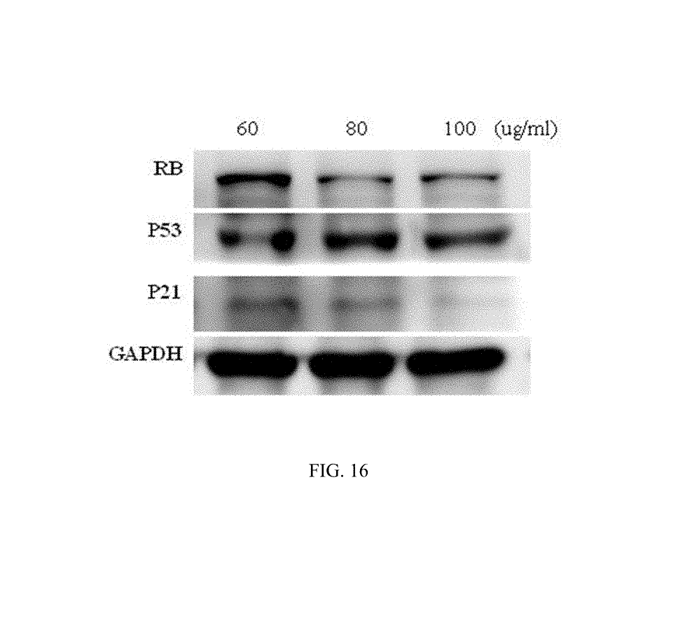

Please refer to FIG. 9A to FIG. 9C. FIG. 9A to FIG. 9C shows the staining result of the SA-β-gal assay of human foreskin fibroblasts, which the cells were cultured on TCPS in the medium containing 100 μg/ml PVA or in the medium containing 100 μg/ml pHEMA. As can be seen in FIG. 9A to FIG. 9C, there is no difference among the cells in the medium with the addition of PVA solution (FIG. 9B), the cells in the medium with the addition of pHEMA solution (FIG. 9C), and the control group without the addition of PVA or pHEMA solution (FIG. 9A) in the staining results of the SA-β-gal assay. As can be seen more clearly in the semi-quantitative results of the SA-β-gal assay of FIG. 10 that the results of the SA-β-gal assay of the 3 groups are almost the same. By the BrdU results of FIG. 11, besides, there is no difference in the results of the BrdU assay of the 3 groups too.

This example found that not all kinds of biocompatible macromolecule materials can be added to the medium to rejuvenate the cells. It is obviously unable to rejuvenate cells by adding PVA or pHEMA solution into the medium, showing the addition of chitosan solution in the medium is not obvious.

### Example 5

The human foreskin fibroblasts were cultured on TCPS and then the chitosan solution was added to the medium (DMEM+10% FBS), and mixed to obtain the composition containing 100 μg/ml chitosan. Next, the pH of the medium containing chitosan was adjusted to pH 7.7 and pH 6.9. The SA-β-gal assay and the BrdU assay were carried out after the cells were cultured at 37° C. with 5% CO₂ for 3 days.

Please refer to FIG. 12A to FIG. 12D. FIG. 12A to FIG. 12D shows the staining result of the SA-β-gal assay of human foreskin fibroblasts, which the cells were cultured in the compositions of the present application at different pH values. As can be seen in FIG. 9A to FIG. 9C, the number of the SA-β-gal-positive cells of the pH 7.7 medium with the addition of chitosan solution (FIG. 12B) or the pH 6.9 medium with the addition of chitosan solution (FIG. 12D) are both less than the cells in the pH 7.7 medium without the addition of chitosan solution (FIG. 12A) and the cells in the pH 6.9 medium without the addition of chitosan solution (FIG. 12C). Furthermore, the number of the SA-β-gal-positive cells of the pH 6.9 medium with the addition of chitosan solution (FIG. 12D) is slightly less than that of the pH 7.7 medium with the addition of chitosan solution (FIG. 12B). By the semi-quantitative results of FIG. 12A to FIG. 12D, the proportion of the SA-β-gal-positive cells of the pH 6.9 medium with the addition of chitosan solution is significantly less than that of the pH 7.7 medium with the addition of chitosan solution (p<0.01).

Please refer to FIG. 14. FIG. 14 shows the BrdU assay result of human foreskin fibroblasts, which the cells were cultured in the compositions of the present application at different pH values. As can be seen in FIG. 14, no matter the medium of pH 7.7 or the medium of pH 6.9, the BrdU absorbance of the cells with the addition of chitosan solution increases significantly compared with those of the cells without the addition of chitosan solution (p<0.01). Besides, the increase of the BrdU absorbance is more significant in the cells of the pH 6.9 medium with the addition of chitosan solution compared with that of the cells of the pH 7.7 medium with the addition of chitosan solution (p<0.01).

This example found that adjusting the composition of the present application to a lower pH (weak acid), such as pH6.9, can achieve better efficacy of rejuvenating cells.

### Example 6

The human foreskin keratinocytes were used in this example, which were obtained in the same manner as in Example 1 except that the keratinocytes were cultured in KSFM containing Bovine Pituitary Extract (BPE) after separating the epidermis from the dermis using 1 mg/ml dispase and collecting the epidermal cells by centrifugation.

The human foreskin keratinocytes were cultured on TCPS and then the chitosan solution was added to the medium (KSFM+BPE), and mixed to form different concentrations of compositions, while the compositions contain 20 μg/ml, 60 μg/ml, and 100 μg/ml of chitosan, respectively. The SA-β-gal assay and the western blot assay for detecting the expression of related proteins RB, P53, and P21 were carried out after the cells were cultured at 37° C. with 5% CO₂ for 3 days.

Please refer to FIG. 15A to FIG. 15F. FIG. 15A to FIG. 15F shows a staining result of the SA-beta-gal assay of human foreskin keratinocytes, which the cells were cultured on TCPS in the compositions containing different chitosan concentrations. As can be seen in FIG. 15A to FIG. 15F, most of the cells in the medium without the addition of chitosan solution (FIG. 15A) are stained blue, whereas for the cells in the medium containing 20 μg/ml (FIG. 15B), 40 μg/ml (FIG. 15C), 60 μg/ml (FIG. 15D), 80 μg/ml (FIG. 15E), and 100 μg/ml (FIG. 15F) chitosan, the number of the SA-β-gal-positive cells decrease with the increase of the chitosan concentrations in the compositions.

Please refer to FIG. 16. FIG. 16 shows the results of RB, p53, and p21 expression of human foreskin fibroblasts using the western blot assay, which the cells were cultured on TCPS in the compositions containing different chitosan concentrations. As can be seen in FIG. 16, the protein expression, whichever the expression of protein RB, p53 or p21, decrease with the increase of the chitosan concentrations in the compositions.

This example found that the method and the composition of the present application can be applied to different types of cell and achieve the same effect of rejuvenating cells. In addition, the method and the composition of the present application can be applied to different types of medium, and different types of medium can be selected for different types of cells. Furthermore, it can be applied to conventional medium for cell culture, or any commercially available cells/tissue medium.

What is claimed is:

1. A method of using chitosan for rejuvenating cells, characterized in that providing a cell to culture in a composition for a suitable period of time, wherein the composition is obtained by adding a chitosan solution to a cultural environment.

2. The method of claim 1, wherein the chitosan concentration of the composition is 10 μg/ml to 200 μg/ml and the chitosan solution comprises 0.05 wt % to 75 wt % of acetic acid.

3. The method of claim 1, wherein the cell is selected from the group consisting of fibroblast, adipose-derived stem cell, mesenchymal stem cell, cruciate ligament cell, synoviocyte, and keratinocyte.

4. The method of claim 1, wherein the pH value of the composition is 6 to 8.

5. The method of claim 4, wherein the pH value of the composition is 6 to 7.

6. The method of claim 1, wherein the suitable period of time is more than 1 day.

7. The method of claim 6, wherein the suitable period of time is 1-7 days.

8. A composition for rejuvenating a cell comprising a chitosan solution in a cultural environment.

9. The composition of claim 8, wherein the chitosan concentration of the composition is 10 μg/ml to 200 μg/ml and the chitosan solution comprises 0.05 wt % to 75 wt % of acetic acid.

10. The composition of claim 8, wherein the pH value of the composition is 6 to 7 or 7 to 8.
